(19) United States
(12) Patent Application Publication
(10) Pub. No.: US 2024/0307144 A1
(43) Pub. Date: Sep. 19, 2024

(54) THROMBECTOMY SYSTEM WITH LOADING BAY DOORS

(71) Applicant: Boston Scientific Scimed, Inc., Maple Grove, MN (US)

(72) Inventors: Laszlo Trent Farago, Hudson, WI (US); Jason W. Sprain, Shoreview, MN (US); Ted Jerome Perron, White Bear Lake, MN (US)

(73) Assignee: Boston Scientific Scimed, Inc., Maple Grove, MN (US)

(21) Appl. No.: 18/604,956

(22) Filed: Mar. 14, 2024

Related U.S. Application Data
(60) Provisional application No. 63/452,517, filed on Mar. 16, 2023.

(51) Int. Cl. A61B 50/13; A61B 50/10

(52) U.S. Cl. CPC A61B 50/13; A61B 2050/105

(57) ABSTRACT

Thrombectomy systems including a console having loading bay doors. An illustrative console may comprise a plurality of removable panels enclosing the internal structure of the console, at least one door assembly, a carriage assembly configured to receive a pump/catheter assembly and disposed within the interior of the console and accessible via the at least one door assembly, and at least one linear actuator coupled to the at least one door assembly. The at least one door assembly may be movable between an open configuration configured to expose an interior of the console and a closed configuration configured to enclose the interior of the console. Linear movement of the at least one linear actuator may be translated to rotational movement of the at least one door assembly.

### CROSS-REFERENCE TO RELATED APPLICATIONS

This application claims the benefit of U.S. Provisional Patent Application Ser. No. 63/452,517, filed Mar. 16, 2023, which is incorporated herein by reference.

### TECHNICAL FIELD

The disclosure is directed to thrombectomy systems. More particularly, the disclosure is directed to a thrombectomy system with doors for enclosing portions of the catheter system.

### BACKGROUND

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be extracted from the vessel through the catheter. Of the known thrombectomy systems and methods, there is an ongoing need to provide alternative configurations of thrombectomy catheters and systems, as well as methods of operating such thrombectomy systems.

### SUMMARY

This disclosure provides design, material, manufacturing method, and use alternatives for medical devices.

In a first example, a console for a thrombectomy system may comprise one or more panels enclosing an internal structure of the console, at least one door assembly, the at least one door assembly movable between an open configuration configured to expose an interior of the console and a closed configuration configured to enclose the interior of the console, a carriage assembly disposed within the interior of the console and accessible via the at least one door assembly, the carriage assembly configured to receive a pump/catheter assembly, and at least one linear actuator coupled to the at least one door assembly. Linear movement of the at least one linear actuator may be translated to rotational movement of the at least one door assembly.

Alternatively or additionally to any of the examples above, in another example, the at least one first door assembly may be configured to open into the interior of console.

Alternatively or additionally to any of the examples above, in another example, the at least one door assembly may be configured to rotate about an outer lateral side thereof.

Alternatively or additionally to any of the examples above, in another example, the at least one door assembly may comprise a door panel and a first door attachment bracket. The first door attachment bracket may be coupled to a first end of the door panel.

Alternatively or additionally to any of the examples above, in another example, the console may further comprise a fixation member coupled to the first door attachment bracket. The fixation member may be configured to be rotatably coupled to a frame of the console.

Alternatively or additionally to any of the examples above, in another example, the console may further comprise a drive hinge coupled to a second end of the door panel.

Alternatively or additionally to any of the examples above, in another example, the drive hinge may be coupled to a bell crank system. The bell crank system may be coupled to the at least one linear actuator.

Alternatively or additionally to any of the examples above, in another example, the bell crank system may comprise a second door attachment bracket, an arm extending radially from the second door attachment bracket, and an actuator rod coupled to the arm.

Alternatively or additionally to any of the examples above, in another example, the console may further comprise at least one magnet disposed adjacent to a first end of the at least one door assembly.

Alternatively or additionally to any of the examples above, in another example, the at least one door assembly may comprise a first door assembly and a second door assembly.

Alternatively or additionally to any of the examples above, in another example, the at least one linear actuator may comprise a first linear actuator coupled to the first door assembly and a second linear actuator coupled to the second door assembly.

Alternatively or additionally to any of the examples above, in another example, the first door assembly and the second door assembly may be configured to be independently operated.

Alternatively or additionally to any of the examples above, in another example, the first door assembly and the second door assembly may be configured to be simultaneously operated.

Alternatively or additionally to any of the examples above, in another example, when the first door assembly and the second door assembly are in the closed configuration, the first door assembly and the second door assembly may define an opening configured to allow a portion of a pump/catheter assembly to pass therethrough.

Alternatively or additionally to any of the examples above, in another example, the console may further comprise a control panel.

Alternatively or additionally to any of the examples above, in another example, the at least one first door assembly may include a first door assembly and a second door assembly, wherein both the first door assembly and the second door assembly may be configured to open into the interior of console.

In another example, a console for a thrombectomy system may comprise a plurality of panels enclosing an internal structure of the console, a first door assembly comprising a door panel, a first door attachment bracket, and a magnet disposed within a cavity of the first door attachment bracket, a second door assembly comprising a door panel, a first door attachment bracket, and a magnet disposed within a cavity of the first door attachment bracket, a first linear actuator coupled to the first door assembly, a second linear actuator coupled to the second door assembly, and a carriage assembly disposed within the interior of the console and accessible via the first door assembly and the second door assembly. The carriage assembly may be configured to receive a pump/catheter assembly. The first linear actuator may be configured to move the first door assembly between an open configuration configured to expose an interior of the console and a closed configuration configured to enclose the interior of the console and the second linear actuator may be configured to move the second door assembly between an open configuration configured to expose an interior of the console and a closed configuration configured to enclose the interior of the console.

Alternatively or additionally to any of the examples above, in another example, the first linear actuator may be coupled to the first door assembly via a first bell crank assembly and the second linear actuator may be coupled to the second door assembly via a second bell crank assembly.

Alternatively or additionally to any of the examples above, in another example, the first bell crank assembly and the second bell crank assembly may each comprise a second door attachment bracket, an arm extending radially from the second door attachment bracket, and an actuator rod coupled to the arm.

Alternatively or additionally to any of the examples above, in another example, the console may further comprise a first drive hinge extending between the first door panel and the first bell crank assembly and a second drive hinge extending between the second door panel and the second bell crank assembly.

In another example, a console for a thrombectomy system a housing enclosing an internal structure of the console, a first door assembly comprising a door panel and a first door attachment bracket, a second door assembly comprising a door panel and a first door attachment bracket, a first linear actuator coupled to the first door assembly, a second linear actuator coupled to the second door assembly, and a carriage assembly disposed within the interior of the console and accessible via the first door assembly and the second door assembly, the carriage assembly configured to receive a pump/catheter assembly. The first door assembly and the second door assembly may each be movable between an open configuration configured to expose an interior of the console and a closed configuration configured to enclose the interior of the console. The first door assembly and the second door assembly may be configured to be moved independently or simultaneously.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative thrombectomy system;

FIG. 2 is a front view of an illustrative thrombectomy console with the doors in a closed configuration;

FIG. 3 is a perspective view of a portion of the thrombectomy console with the door assemblies in an open configuration;

FIG. 4A is an exploded perspective view of the first door assembly and the first actuation assembly;

FIG. 4B is an enlarged exploded front perspective view of the drive hinge and the actuation assembly;

FIG. 4C is an enlarged exploded side perspective view of the drive hinge and the actuation assembly;

FIG. 5 is a perspective view of a portion of the thrombectomy console with the first door assembly in an open configuration and the second door assembly in a closed configuration;

FIG. 6 is an illustrative control screen for operating the door assemblies of the thrombectomy console; and

FIG. 7 is another illustrative control screen for operating the door assemblies of the thrombectomy console.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Thrombectomy catheters and systems may be used to remove thrombus, plaques, lesions, clots, etc. from veins or arteries. The control console or drive unit of a thrombectomy system may enclose portions of the thrombectomy catheter. Doors may be provided which are selectively opened to allow the user to place the single use device within the loading bay of a console and closed to allow for the safe operation of the system. Disclosed herein is a loading bay door system which allows for greater user control and increased safety.

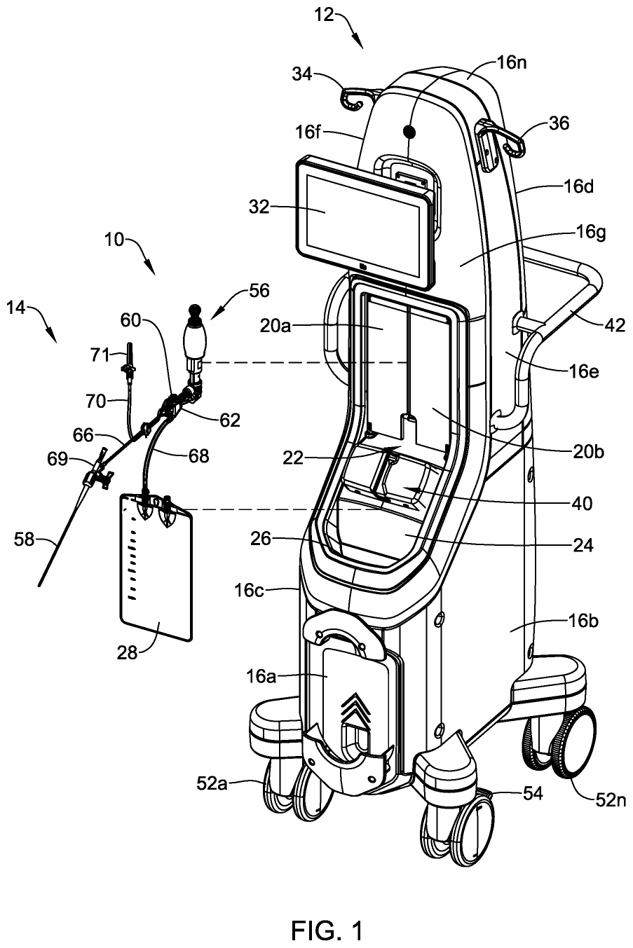

FIG. 1 is a perspective view of an illustrative thrombectomy system 10. The thrombectomy system 10 may include a control console 12, including a drive unit, and a pump/catheter assembly 14. In some instances, the pump/catheter assembly 14 may be a single use device in which a new pump/catheter assembly 14 may be used with the console 12 for each medical procedure. The console 12 may include a housing enclosing the internal structure of the console 12. Shown on the console 12 are a plurality of removable panels 16a-16n about and along the console 12 enclosing the internal structure of the console 12. An illustrative console 12 is described in commonly assigned U.S. Pat. No. 7,935,077, titled THROMBECTOMY CATHETER DEPLOYMENT SYSTEM, the disclosure of which is hereby incorporated by reference. Centrally located in the console 12 and aligned to the lower region of the panel 16g may be automatically opening loading bay door assemblies 20a, 20b which open to expose the interior of the console 12 to provide access to a carriage assembly 22. The carriage assembly 22, which may accommodate components of the pump/catheter assembly 14 is shown accessible via opening the closed door assemblies 20a, 20b. The console 12 may include a catch basin for collecting fluid leakage from the components of the pump/catheter assembly 14. For example, a drip tray 24 is shown located on the front of the console 12 extending from below the carriage assembly 22 toward the panel 16a. In some instances, the drip tray 24 may be removable. Other configurations of catch basins are also contemplated. The drip tray 24 and/or a receptacle 26 may collectively support and accommodate an effluent collection bag, such as effluent collection bag 28 of the pump/catheter assembly 14. In other instances, the console 12 may include a different structure, such as a hook for hanging the effluent collection bag 28 from, or a shelf for setting the effluent collection bag 28 on. The effluent waste tube 68 may also be positioned in the roller pump 40 between the tube guides with the effluent collection bag 28 connected to the effluent waste tube 68. The effluent collection bag 28 may be suitably positioned for collecting effluent during the medical procedure. Pump rollers (not shown) of the roller pump 40 may rotatably engage the effluent waste tube 68 to control effluent fluid flow through the effluent waste tube 68 to the effluent collection bag 28.

In instances where the carriage assembly 22 is movable, a carriage assembly activation switch (not explicitly shown) may be provided with the console 12, such as located on panel 16g, to selectively position the carriage assembly 22 inwardly or outwardly. In other instances, the carriage assembly 22 may be positioned using a control panel and/or user interface 32. A user interface 32, including memory and/or processing capabilities, may be provided with the console 12, such as located at the upper region of the console 12 between the upper regions of the upper side panels 16e and 16f. The user interface 32 may be a guided user interface (GUI) including a touch screen display to allow a user to provide input to the user interface 32 and view information on a same display screen. However, this is not required. In other instances, the user input may be separate from the display screen. Saline bag hooks 34 and 36 may extend through the panels 16e and 16f to hang saline bags therefrom. The console 12 may include a handle 42 as well as a plurality of wheels 52a-52n and brake pedals 54 for wheel lockage to assist in maneuvering the console 12 by medical personnel.

The pump/catheter assembly 14, which may be a disposable single-use device, is shown unattached from the console 12. The pump/catheter assembly 14 includes a pump 56 and a thrombectomy catheter 58. During use, a portion of the pump/catheter assembly 14 may be secured within a portion of the console 12. Other components included in the pump/catheter assembly 14 may include a bubble trap 60 attached to the pump 56, a connection manifold assembly 62 connected to the bubble trap 60, an effluent return tube 66 connected between the connection manifold assembly 62 and the thrombectomy catheter 58, a high-pressure fluid supply tube (not explicitly shown) attached between the output of the pump 56 and the thrombectomy catheter 58 which may be coaxially arranged inside the effluent return tube 66, a transition fixture 69 between the distal end of the effluent return tube 66 and the proximal end of the thrombectomy catheter 58, an effluent waste tube 68 connecting the effluent collection bag 28 to the connection manifold assembly 62, and a fluid supply tube 70 having a bag spike 71 connecting a fluid supply bag (e.g., a saline bag) to the connection manifold assembly 62. The fluid supply tube 70 may be in fluid communication with the interior of the bubble trap 60 to provide fluid from the fluid supply bag to the pump 56 and then to the thrombectomy catheter 58 through the high-pressure fluid supply tube.

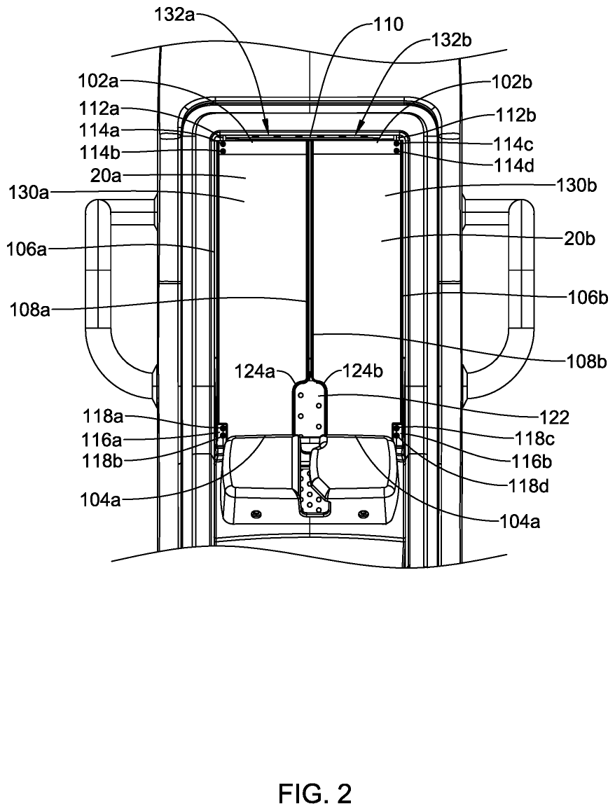

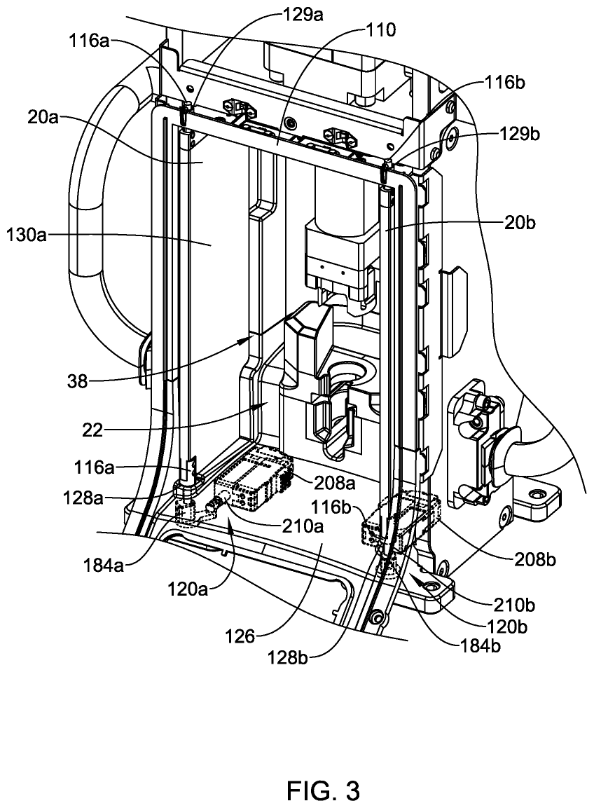

FIG. 2 is a front view of an upper portion of the console 12 with the door assemblies 20a, 20b in a closed configuration. Each door assembly 20a, 20b may include a first end 102a, 102b, a second end 104a, 104b, an outer lateral side 106a, 106b, and an inner lateral side 108a, 108b. The outer lateral sides 106a, 106b and inner laterals sides 108a, 108b may each extend from the first end 102a, 102b to the second end 104a, 104b. Each door 30 assembly 20a, 20b may each be pivotably coupled to a frame 110 of the console 12 adjacent to the outer lateral side 106a, 106b thereof. For example, the first door assembly 20a may include a pin 112a fixedly coupled adjacent to the first end 102a and the outer lateral side 106a via one or more fixation members 114a, 114b, such as, but not limited to set screws, screws, bolts, rivets, etc. The pin 112a may extend away from the first end 102a of the first door assembly 20a in a direction opposite from the second end 104a. The pin 112a may be rotatably received within an opening or aperture 129a (see, for example, FIG. 3) of the frame 110. Thus, the pin 112a may be configured to rotate as the first door assembly 20a pivots between an open configuration (FIG. 3) and a closed configuration (FIG. 2). Similarly, the second door assembly 20b may include a pin 112b fixedly coupled adjacent to the first end 102b and the outer lateral side 106b via one or more fixation members 114c, 114d, such as, but not limited to set screws, screws, bolts, pins, etc. The pin 112b may extend away from the first end 102b of the door assembly 20b in a direction opposite from the second end 104b. The pin 112b may be rotatably received within an opening or aperture 129b (see, for example, FIG. 3) of the frame 110. Thus, the pin 112b may be configured to rotate as the second door assembly 20b pivots between an open configuration (FIG. 3) and a closed configuration (FIG. 2).

The first door assembly 20a may also be fixedly coupled to a drive hinge 116a adjacent to the second end 104a and the outer lateral side 106a thereof via one or more fixation members 118a, 118b, such as, but not limited to, set screws, screws, bolts, pins, etc. As will be described in more detail herein, the drive hinge 116a may be coupled to an actuation assembly 120a (see, for example, FIG. 3) configured to move the first door assembly 20a between a closed configuration and an open configuration. Similarly, the second door assembly 20b may also be fixedly coupled to a drive hinge 116b adjacent to the second end 104b and the outer lateral side 106b thereof via one or more fixation members 118b, 118b, such as, but not limited to, set screws, screws, bolts, pins, etc. As will be described in more detail herein, the drive hinge 116b may be coupled to an actuation assembly 120b (see, for example, FIG. 3) configured to move the door assembly 20b between a closed configuration and an open configuration.

The width of the door assemblies 20a, 20b may be smaller adjacent to the second end 104a, 104b thereof than a width of the door assemblies 20a, 20b adjacent to the first end 102a, 102b thereof. For example, the width of the door assemblies 20a, 20b may transition from the first width to the second smaller width at a curved region 124a, 124b of the inner lateral sides 108a, 108b. The first and second door assemblies 20a, 20b may be mirror images of one another such that the curved regions 124a, 124b are positioned at similar axial locations. The reduction in the width of the door assemblies 20a, 20b may define an opening 122 for allowing components of the thrombectomy catheter 58 to extend between an interior or loading bay 38 (FIG. 3) of the console 12 and an exterior of the console 12. It is further contemplated that the opening 122 may be sized and shaped to allow the door assemblies 20a, 20b to be opened and closed when a pump/catheter assembly 14 is assembled with the carriage assembly 22.

FIG. 3 is a perspective view of a portion of the console 12 with the door assemblies 20a, 20b in an open configuration. The door assemblies 20a, 20b may each be configured to rotate or hinge inward about an axis that extends through the pin 112a, 112b and the drive hinge 116a, 116b into a loading bay or interior 38. This may locate any pinch points within the loading bay or interior 38 providing pinch point protection to the user. It is further contemplated that inwardly opening door assemblies 20a, 20b may allow for better user access to the loading bay 38 of the console 12. In some examples, the interior walls of the console 12 may include recesses sized and shaped to allow the door assemblies 20a, 20b to be positioned therein when the door assemblies are in the open configuration. While the door assemblies 20a, 20b are described as rotating into the loading bay 38 as they open, it is contemplated that in some cases, the door assemblies 20a, 20b may rotate away from the loading bay 38, or outward, as they open.

As described above, the drive hinges 116a, 116b may each be coupled to an individual actuation assembly 120a, 120b. This may allow the door assemblies 20a, 20b to be operated individually or substantially simultaneously, as will be described in more detail herein. The drive hinges 116a, 116b may pass through apertures 128a, 128b extending though a bottom surface 126 of the frame 110. A portion of the drive hinge 116a, 116b may be coupled to the actuation assembly 120a, 120b.

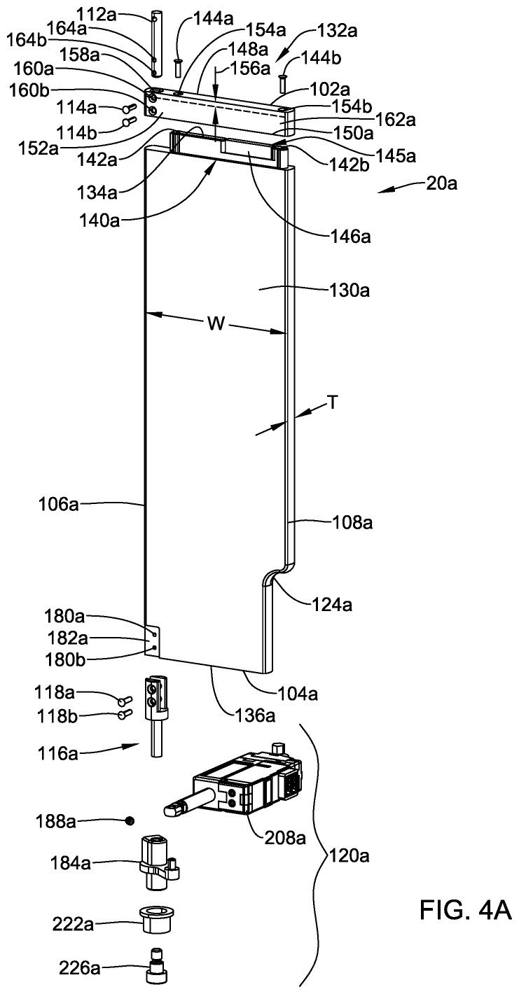

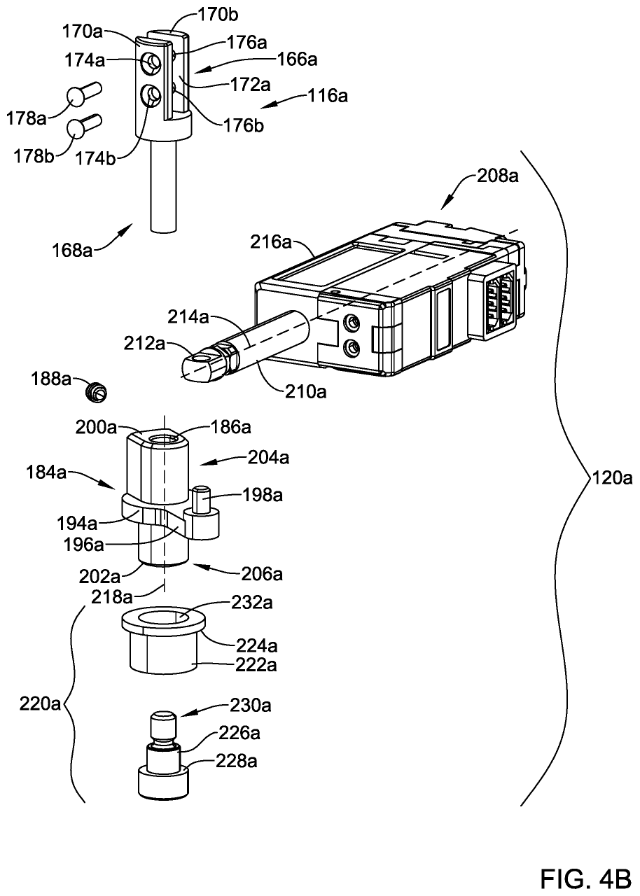

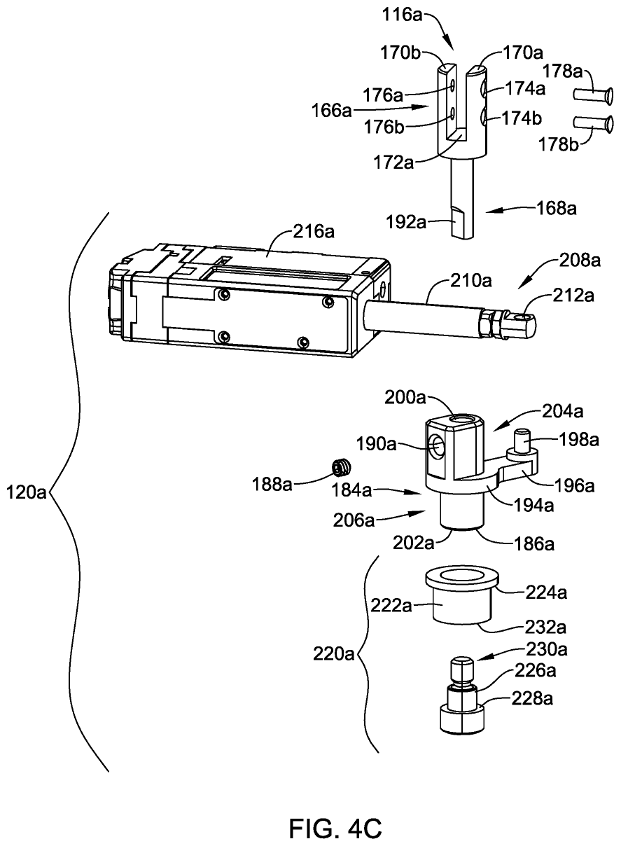

FIG. 4A is an exploded perspective view of the first door assembly 20a and the first actuation assembly 120a. It should be understood that the second door assembly 20b and the second actuation assembly 120b include all of the same components as the first door assembly 20a and the first actuation assembly 120a in a mirror image configuration. The first door assembly 20a may include a door panel 130a and a first door attachment bracket 132a. The first door attachment bracket 132a may include the pin 112a for hingedly coupling the door assembly 20a to the console 12 as well as one or more fixation members. The first door attachment bracket 132a may have a width and thickness similar to a width W and thickness T of the door panel 130a. The door panel 130a may extend from a first end 134a to a second end 136a. In some examples, the door panel 130a may include a first end region 140a that has a reduced width and thickness than the width W and thickness T of an intermediate region of the door panel 130a. This may allow the first door attachment bracket 132a to be disposed over the first end region 140a of the door panel 130a while maintaining relatively uniform outer dimensions of the door assembly 20a. The first end region 140a of the door panel 130a may include a first aperture 142a and a second aperture 142b each for receiving a fixation member 144a, 144b therein. The fixation members 144a, 144b may include, but are not limited to set screws, screws, bolts, pins, etc. Generally, the fixation members 144a, 144b may be configured to secure the first door attachment bracket 132a to the door panel 130a. The first end region 140a of the door panel 130a may further include a magnet 146a disposed within a recess 145a formed in the first end region 140a. A hall effect sensor (not explicitly shown) may be positioned in the frame 110 of the console 12 adjacent to the magnet 146a when the door assembly 20a is in the closed configuration. The hall effect sensor may detect the presence of the magnet 146a to determine whether the door assembly 20a is open or closed. It is contemplated that a procedure may not begin until both door assemblies 20a, 20b are in the closed configuration.

The door attachment bracket 132a may be secured to the door panel 130a adjacent to a first end 134a of the door panel 130a. The first door attachment bracket 132a may extend from a first generally closed end 148a to a second generally open end 150a. For example, the second end 150a may be configured to be disposed over the first end region 140a of the door panel 130a. For example, the first door attachment bracket 132a may include a cavity 152a extending from the second end 150a towards the first end 148a, but not extending through an entirety of the height of the first door attachment bracket 132a. When the first door attachment bracket 132a is assembled with the door panel 130a, the first door attachment bracket 132a may be secured to the door panel 130a via the one or more fixation members 144a, 144b. For example, the fixation members 144a, 144b may extend through a first aperture 154a and a second aperture 154b of the first door attachment bracket 132a, respectively. The first and second apertures 154a, 154b may extend from the first end 148a and through a wall 156a thereof. The fixation members 144a, 144b may also extend into the apertures 142a, 142b at the first end 134a of the door panel 130a. When the first door attachment bracket 132a is assembled with the door panel 130a, the magnet 146a may be disposed within the cavity 152a of the first door attachment bracket 132a.

The first door attachment bracket 132a may further include a through hole or aperture 158a extending from the first end 148a to the second end 150a. The through hole 158a may be configured to receive the pin 112a therein. The first door attachment bracket 132a may further include a third aperture 160a and a fourth aperture 160b extending through a front surface 162a thereof. The third and fourth apertures 160a, 160b may extend through the front surface 162a adjacent to the through hole 158a. The third and fourth apertures 160a, 160b may be configured to receive the fixation members 114a, 114b to fixedly couple the pin 112a to the first door attachment bracket 132a and thus the first door assembly 20a. It is contemplated that the fixation members 114a, 114b may be configured to engage apertures 164a, 164b formed in a second end region of the pin 112a. Alternatively, the fixation members 114a, 114b may be configured to frictionally engage an outer surface of the pin 112a.

The drive hinge 116a may be secured to the door panel 130a adjacent the second end 136a thereof. Referring additionally to FIGS. 4B and 4C which illustrate an enlarged exploded front perspective view and an enlarged exploded side perspective view, respectively, of the drive hinge 116a and the actuation assembly 120a, the drive hinge 116a may include a first end region 166a and a second end region 168a. The first end region 166a may have a generally “U” shaped configuration including a first arm 170a, a second arm 170b, and a channel 172a therebetween. Each arm 170a, 170b may include a first aperture 174a, 176a and a second aperture 174b, 176b extending through a thickness thereof. The first apertures 174a, 176a may each be configured to receive a first fixation member 178a therethrough and the second apertures 174b, 176b may each be configured to receive a first fixation member 178b therethrough. The fixation members 178a, 178b may be configured to extend through apertures 180a, 180b (see, for example, FIG. 4A) extending through a thickness of the door panel 130a. The apertures 180a, 180b may be formed through a corner of the door panel 130a adjacent to the outer lateral side 106a and the second end 104a of the door assembly 20a in a corner region 182a having a reduced thickness relative to the thickness T of an intermediate region of the first door panel 130a adjacent to the inner lateral side 108a and the second end 104a of the door assembly 20a. The corner region 182a may be sized and shaped to be assembled within the channel 172a of the drive hinge 116a. When the corner region 182a is assembled within the channel 172a of the drive hinge 116a, the first apertures 174a, 176a of the arms 170a, 170b may be aligned with the first aperture 180a of the corner region 182a. Similarly, the second apertures 174b, 176b of the arms 170a, 170b may be aligned with the second aperture 180b of the corner region 182a. When assembled, the fixation members 178a, 178b may be inserted into the apertures 174a, 174b of the first arm 170a, through the apertures 180a, 180b of the corner region 182a, and into the apertures 176a, 176b of the second arm 170b to secure the drive hinge 116a to the door assembly 20a.

The second end region 168a of the drive hinge 116a may be configured to be disposed within a lumen 186a of a second door attachment bracket 184a. The second end region 168a of the drive hinge 116a may be secured to the second door attachment bracket 184a via a fixation member 188a such as, but not limited to, a set screw, screw, bolt, pin, etc. The fixation member 188a may extend through an aperture 190a extending through a side wall of the second door attachment bracket 184a to the lumen 186a thereof to frictionally engage a flattened coupling surface 192a of the drive hinge 116a.

The second door attachment bracket 184a may further include radially extending region 194a with an arm 196a extending radially from the radially extending region 194a. The radially extending region 194a may be positioned between a first end 200a and a second end 202a of the second door attachment bracket 184a. A first end region 204a of the second door attachment bracket 184a may be disposed above the radially extending region 194a and a second end region 206a of the second door attachment bracket 184a may be disposed below the radially extending region 194a. A laterally extending protrusion or pin 198a may extend upwards (e.g., towards the first end 200a of the second door attachment bracket 184a) from an upper surface of the arm 196a. The pin 198a may have a longitudinal axis that extends generally parallel to a longitudinal axis 218a of the lumen 186a of the second door attachment bracket 184a.

The pin 198a may be configured to be received within a mating aperture 212a extending though an actuator rod 210a of an electric linear actuator 208a. Collectively, the arm 196a, the pin 198a, and the actuator rod 210a of the linear actuator 208a may form a bell crank system. The actuator rod 210a may be actuatable along a longitudinal axis 214a thereof. In the illustrated embodiment, the actuator rod 210a is shown in an extended configuration. In the illustrated embodiment, when the actuator rod 210a is in the extended configuration, the door assembly 20a is in the closed configuration. However, this is not required. In some cases, the door assembly 20a may be in the open configuration when the actuator rod 210a is in the extended configuration. The actuator rod 210a may be retracted into the housing 216a of the linear actuator 208a. As the actuator rod 210a is retracted into the housing 216a of the linear actuator 208a, the actuator rod 210a pulls the pin 198a and translates linear motion of the actuator rod 210a to rotational movement of the second door attachment bracket 184a. The torque is transferred to the door assembly 20a from the second door attachment bracket 184a via the drive hinge 116a to move the door assembly 20a from a closed configuration to an open configuration. For example, as the actuator rod 210a is actuated, the second door attachment bracket 184a pivots about the longitudinal axis 218a of the lumen 186a of the second door attachment bracket 184a. The linear actuation of the actuator rod 210a may be reversed to extend the actuator rod 210a and move the door assembly 20a from an open configuration to a closed configuration.

The second door attachment bracket 184a may be rotatably coupled to the console 12 via a coupling mechanism 220a. The coupling mechanism 220a may be configured to extend through an opening in an internal plate of the console 12 (e.g., located under the bottom surface 126 of the frame 110). Generally, a first portion 222a of the coupling mechanism 220a may include a first surface 224a configured to engage a first side of an internal plate of the console 12 and a second portion 226a of the coupling mechanism 220a may include a second surface 228a configured to engage a second side of an internal plate of the console 12. For example, the first surface 224a may have a greater diameter than a diameter of the opening in the internal plate of the console 12 adjacent the first side thereof. Similarly, the second surface 228a may have a greater diameter than a diameter of the opening in the internal plate of the console 12 adjacent the second side thereof. The diameter of the opening in the internal plate may vary along the length thereof. The internal plate of the console 12 may be positioned between the first portion 222a and the second portion 226a of the coupling mechanism 220a. A first end region 230a of the second portion 226a of the coupling mechanism 220a may be disposed within the lumen 186a of the second door attachment bracket 184a. In some cases, at least part of the first end region 230a may frictionally engage the inner surface of the lumen 186a to couple the second door attachment bracket 184a to the second portion 226a of the coupling mechanism 220a. Thus, the second portion 226a of the coupling mechanism 220a may rotate with the second door attachment bracket 184a. The second end region 206a of the second door attachment bracket 184a may be rotationally disposed within an aperture 232a of the first portion 222a of the coupling mechanism 220a. Thus, the first portion 222a of the coupling mechanism 220a may remain stationary as the second door attachment bracket 184a is rotated. Alternatively, the first portion 222a of the coupling mechanism 220a may rotate as the second door attachment bracket 184a is rotated.

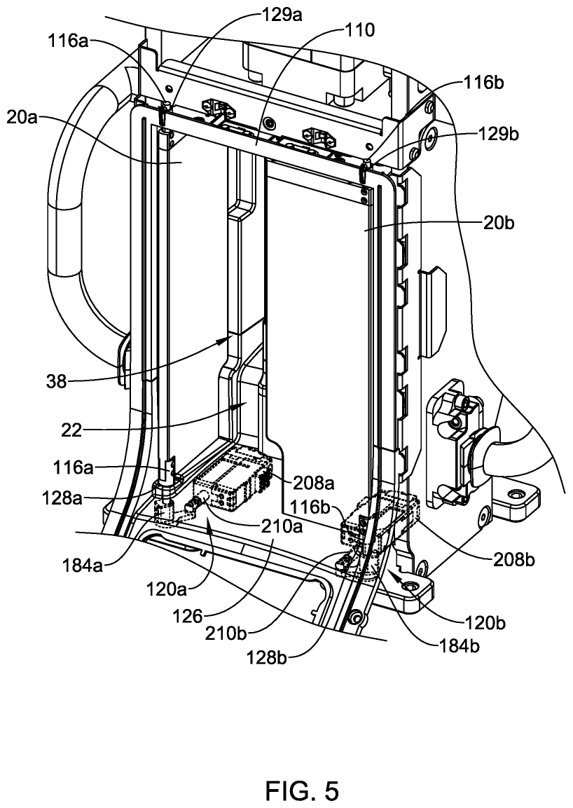

FIG. 5 is a perspective view of a portion of the console 12 with the first door assembly 20a in an open configuration and the second door assembly 20b in a closed configuration. The door assemblies 20a, 20b may each be coupled to their own respective actuation assemblies 120a, 120b. Thus, the door assemblies 20a, 20b may be actuated independently of one another or actuated substantially simultaneously to one another. In other words, in one or more modes of operation, the door assemblies 20a, 20b may be actuated independently, such as during a maintenance and/or cleaning mode where one door assembly 20a is in the closed position while the other door assembly 20b is in the open position. In one or more other modes of operation, such as in a procedure setup mode, the door assemblies 20a, 20b may be actuated simultaneously to open and/or close the door assemblies 20a, 20b.

In the illustrated embodiment of FIG. 5, the first door assembly 20a has been opened. To open the first door assembly 20a, the user may enter a command via the user interface 32. The control module of the console 12 may then transmit a command to the linear actuator 208a to retract the actuator rod 210a to open the first door assembly 20a. It is contemplated that if the first door assembly 20a is configured to move outward, the linear actuator 208a may extend the actuator rod 210a to open the first door assembly 20a. As can be seen in FIG. 5, the actuator rod 210a is shown in the retracted configuration within the housing 216a of the linear actuator 208a. As the actuator rod 210a is retracted, the second door attachment bracket 184a is rotated or pivoted about an axis that extends through the pin 112a and the drive hinge 116a to move the door assembly 20a into the loading bay 38 to the open configuration. As can be seen the drive hinge 116a of the first door assembly 20a is rotated about 90° from the closed configuration (see, for example, FIG. 2). To open the second door assembly 20b, the user may similarly enter a command via the user interface 32. The control module of the console 12 may then transmit a command to the linear actuator 208b to retract the actuator rod 210b to open the second door assembly 20b (see, for example, FIG. 3). It is contemplated that if the second door assembly 20b is configured to move outward, the linear actuator 208b may extend the actuator rod 210b to open the second door assembly 20b. As the actuator rod 210b is retracted, the second door attachment bracket 184b is rotated or pivoted about an axis that extends through the pin 112b and the drive hinge 116b to move the door assembly 20b to the open configuration. When open (FIG. 3) the drive hinge 116b of the second door assembly 20b is rotated about 90° from the closed configuration (see, for example, FIG. 2). In some cases, the door assemblies 20a, 20b may be opened individually to allow the interior surfaces of the door assemblies 20a, 20b to be cleaned.

In some cases, a command may be entered via the user interface 32 to open and/or close both door assemblies 20a, 20b at the same time (i.e., simultaneously). For example, in some embodiments, the door assemblies 20a, 20b may be opened at the same to load the pump/catheter assembly 14 into the carriage assembly and the door assemblies 20a, 20b may be closed at the same time to begin the procedure.

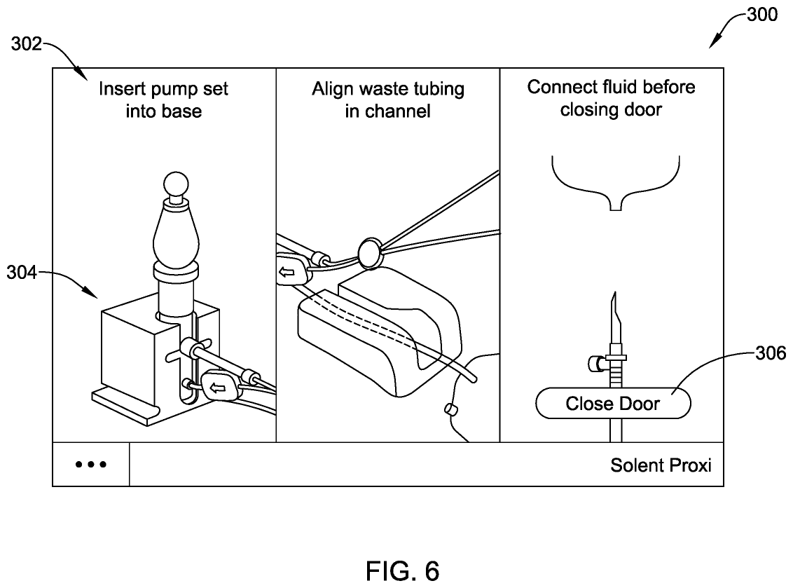

The user interface 32 may provide set-up and/or operational instructions to the user. FIG. 6 is a schematic view of an illustrative control screen 300 for setting up the thrombectomy system 10. For example, the control screen 300 may provide plain language instructions 302 and/or pictorial instructions 304 to assemble the pump/catheter assembly 14 with the console 12. Once the pump/catheter assembly 14 has been assembled with the console 12, the user may close the door assemblies 20a, 20b. This may be accomplished by actuation, for example, a “close door” button or region 306 on the control screen 300. The sensors, such as hall effect sensors or proximity sensors, may be used to determine if the door assemblies 20a, 20b have fully closed. If the door assemblies 20a, 20b do not fully close, a pop-up error or screen may be displayed on the user interface 32. After a predetermined length of time, the door assemblies 20a, 20b will reopen if the error is not cleared. In some cases, an additional pop-up or error screen may instruct the user to verify the path of the door assemblies 20a, 20b is clear. It is contemplated that the console 12 may be precluded from operating the pump/catheter assembly 14 until the door assemblies 20a, 20b are closed and in the closed position.

Once the door assemblies 20a, 20b are closed, the roller pump 40 may be closed. The actuator (not explicitly shown) may be lowered to engage the pump 56 of the pump/catheter assembly 14. It is contemplated that one or more steps of the set-up procedure may be prevented or locked out if the door assemblies 20a, 20b are not in the closed configuration. Once the set-up procedure is complete, the pump/catheter assembly 14 may be operated to remove thrombus, plaques, lesions, clots, etc. from veins or arteries. It is further contemplated that operation of the pump/catheter assembly 14 may be prevented if the door assemblies 20a, 20b are not in the closed configuration.

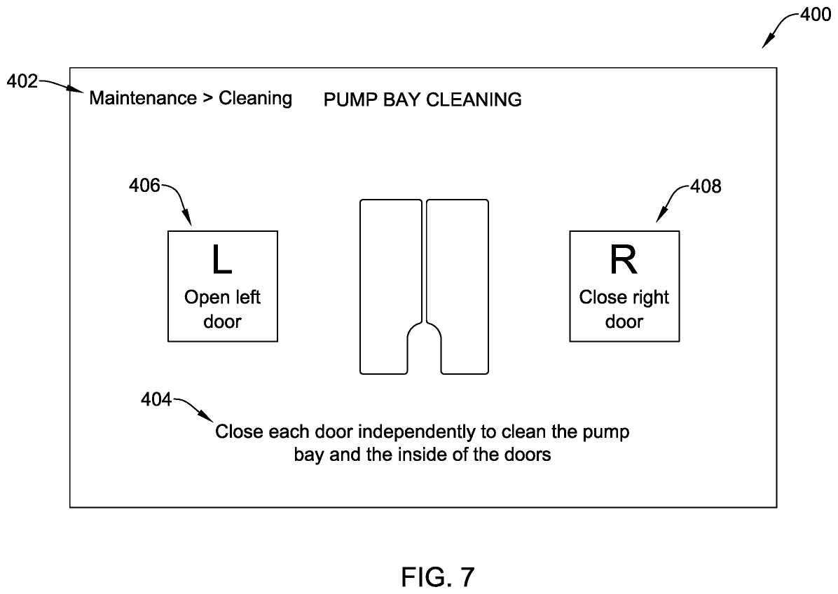

Once the procedure is complete, the user may use the user interface to enter a maintenance mode. FIG. 7 is a schematic view of an illustrative control screen 400 for cleaning the loading bay 38 after a procedure, or as otherwise necessary. For example, from a home screen, the user may first select a maintenance mode and once in the maintenance mode the user may select cleaning, as shown at the navigation tree 402. The user may be provided with plain language instructions 404. The control screen 400 may further include a first button or touch region 406 for closing the left or first door assembly 20a and a second button or touch region 408 for closing the right or second door assembly 20b. The user may select individual door assemblies 20a, 20b to allow the interior of the door assemblies 20a, 20b to be cleaned. It is contemplated that when a user selects one of the touch regions 406, 408 the opposite door assembly 20a, 20b will be closed. For example, if the user selects the first touch region 406 to open the first door assembly 20a, the second door assembly 20b may be closed if it is open.

It is contemplated that the user interface 32 may include additional control screens for setting up the thrombectomy system 10 and/or controlling the operation of the door assemblies 20a, 20b. In some examples, the user interface 32 may allow the user to adjust a speed at which the door assemblies 20a, 20b open. In some cases, the door assemblies 20a, 20b may be configured to open at a same speed. In other examples, the door assemblies 20a, 20b may be configured to operate at different speeds. It is contemplated that the speed at which the door assemblies 20a, 20b open and/or close may be based, at least in part, on how quickly the actuator rod 210a, 210b of the linear actuator 208a, 208b is moved.

The materials that can be used for the various components of the thrombectomy catheter, pump/catheter assembly, and/or other devices disclosed herein may include those commonly associated with medical devices. For simplicity purposes, the following discussion makes reference to the pump/catheter assembly and its related components. However, this is not intended to limit the devices and methods described herein, as the discussion may be applied to other similar devices, tubular members and/or components of tubular members or devices disclosed herein.

The various components of the devices/systems disclosed herein may include a metal, metal alloy, polymer (some examples of which are disclosed herein), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylenc (PP), polyvinylchloride (PVC), polyether-ester (for example, ARNITEL® available from DSM Engineering Plastics), ether ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of the pump/catheter assembly and its related components may be doped with, made of, or otherwise include a radiopaque material. Radiopaque materials are understood to be materials capable of producing a relatively bright image on a fluoroscopy screen or another imaging technique during a medical procedure. This relatively bright image aids the user of the pump/catheter assembly and its related components in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the pump/catheter assembly and its related components to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A console for a thrombectomy system, the console comprising:
one or more panels enclosing an internal structure of the console;
at least one door assembly, the at least one door assembly movable between an open configuration configured to expose an interior of the console and a closed configuration configured to enclose the interior of the console;
a carriage assembly disposed within the interior of the console and accessible via the at least one door assembly, the carriage assembly configured to receive a pump/catheter assembly; and
at least one linear actuator coupled to the at least one door assembly;
wherein linear movement of the at least one linear actuator is translated to rotational movement of the at least one door assembly.

2. The console of claim 1, wherein the at least one first door assembly includes a first door assembly and a second door assembly, wherein both the first door assembly and the second door assembly are configured to open into the interior of console.

3. The console of claim 1, wherein the at least one door assembly is configured to rotate about an outer lateral side thereof.

4. The console of claim 1, wherein the at least one door assembly comprises a door panel and a first door attachment bracket, the first door attachment bracket coupled to a first end of the door panel.

5. The console of claim 4, further comprising a fixation member coupled to the first door attachment bracket, the fixation member configured to be rotatably coupled to a frame of the console.

6. The console of claim 4, further comprising a drive hinge coupled to a second end of the door panel.

7. The console of claim 6, wherein the drive hinge is coupled to a bell crank system, the bell crank system coupled to the at least one linear actuator.

8. The console of claim 7, wherein the bell crank system comprises a second door attachment bracket, an arm extending radially from the second door attachment bracket, and an actuator rod coupled to the arm.

9. The console of claim 1, further comprising at least one magnet disposed adjacent to a first end of the at least one door assembly.

10. The console of claim 1, wherein the at least one door assembly comprises a first door assembly and a second door assembly.

11. The console of claim 10, wherein the at least one linear actuator comprises a first linear actuator coupled to the first door assembly and a second linear actuator coupled to the second door assembly.

12. The console of claim 10, wherein the first door assembly and the second door assembly are configured to be independently operated.

13. The console of claim 10, wherein the first door assembly and the second door assembly are configured to be simultaneously operated.

14. The console of claim 10, wherein when the first door assembly and the second door assembly are in the closed configuration, the first door assembly and the second door assembly define an opening configured to allow a portion of a pump/catheter assembly to pass therethrough.

15. The console of claim 1, further comprising a control panel.

16. A console for a thrombectomy system, the console comprising:
a plurality of panels enclosing an internal structure of the console;
a first door assembly comprising a door panel, a first door attachment bracket, and a magnet disposed within a cavity of the first door attachment bracket;
a second door assembly comprising a door panel, a first door attachment bracket, and a magnet disposed within a cavity of the first door attachment bracket;
a first linear actuator coupled to the first door assembly;
a second linear actuator coupled to the second door assembly; and
a carriage assembly disposed within the interior of the console and accessible via the first door assembly and the second door assembly, the carriage assembly configured to receive a pump/catheter assembly;
wherein the first linear actuator is configured to move the first door assembly between an open configuration configured to expose an interior of the console and a closed configuration configured to enclose the interior of the console and the second linear actuator is configured to move the second door assembly between an open configuration configured to expose an interior of the console and a closed configuration configured to enclose the interior of the console.

17. The console of claim 16, wherein the first linear actuator is coupled to the first door assembly via a first bell crank assembly and the second linear actuator is coupled to the second door assembly via a second bell crank assembly.

18. The console of claim 17, wherein the first bell crank assembly and the second bell crank assembly each comprise a second door attachment bracket, an arm extending radially from the second door attachment bracket, and an actuator rod coupled to the arm.

19. The console of claim 18, further comprising a first drive hinge extending between the first door panel and the first bell crank assembly and a second drive hinge extending between the second door panel and the second bell crank assembly.

20. A console for a thrombectomy system, the console comprising:
a housing enclosing an internal structure of the console;
a first door assembly comprising a door panel and a first door attachment bracket;
a second door assembly comprising a door panel and a first door attachment bracket;
a first linear actuator coupled to the first door assembly;
a second linear actuator coupled to the second door assembly; and
a carriage assembly disposed within the interior of the console and accessible via the first door assembly and the second door assembly, the carriage assembly configured to receive a pump/catheter assembly;
wherein the first door assembly and the second door assembly are each movable between an open configuration configured to expose an interior of the console and a closed configuration configured to enclose the interior of the console; and
wherein the first door assembly and the second door assembly are configured to be moved independently or simultaneously.
